(19) United States
(12) Patent Application Publication
(10) Pub. No.: US 2022/0273944 A1
(43) Pub. Date: Sep. 1, 2022

(54) CARDIAC STIMULATION SYSTEM

(71) Applicant: MAXWELL BIOMEDICAL, INC., Solana Beach, CA (US)

(72) Inventors: Randell L. Werneth, San Diego, CA (US); David Zarbatany, Laguna Niguel, CA (US); Ricardo David Roman, Chula Vista, CA (US); Marcus Julian, Vista, CA (US); R. Maxwell Flaherty, Topsfield, MA (US); J. Christopher Flaherty, Auburndale, FL (US)

(21) Appl. No.: 17/637,877

(22) PCT Filed: Sep. 4, 2020
(86) PCT No.: PCT/US20/49349

§ 371 Date: Feb. 24, 2022

Related U.S. Application Data
(60) Provisional application No. 62/895,655, filed on Sep. 4, 2019.

(51) Int. Cl. A61N 1/05; A61N 1/378

(52) U.S. Cl. CPC A61N 1/0563; A61N 1/3787; A61N 2001/0585

(57) ABSTRACT

Provided herein are systems for stimulating cardiac tissue of a patient. The systems include: a pulse generator having a first transmission element for delivering wireless power; a stimulation assembly having a flexible substrate, a second transmission element for receiving the wireless power from the first transmission element of the pulse generator, one or more electrodes attached to the substrate for delivering electrical energy to cardiac tissue, and one or more microcircuits attached to the substrate for delivering electrical energy to the one or more electrodes; and an algorithm having a fibrillation detection algorithm for determining when the one or more electrodes deliver the energy to the cardiac tissue.

### RELATED APPLICATIONS

The present application claims priority to U.S. Provisional Patent Application Ser. No. 62/895,655, entitled “Multi-site micro pacing circuits and algorithms, integrated into a Venously Placed Stent Assembly”, filed Sep. 4, 2019, the content of which is incorporated herein by reference in its entirety for all purposes.

### FIELD OF THE INVENTION

The present inventive concepts relate generally to implantable systems and methods for performing targeted tissue pacing in a patient. In particular, the present inventive concepts present an expandable scaffold and pulse generator which deliver pacing energy to a patient suffering from atrial fibrillation and/or other arrhythmias for the purpose of converting the patient back to normal sinus rhythm.

BACKGROUND

Various types of implantable cardiac stimulation devices are presently available and used for delivering various types of cardiac stimulation therapy in the treatment of cardiac arrhythmias. The two most common types, which are in widespread use, are pacemakers and implantable cardioverter defibrillators (ICDs). Pacemakers generally produce relatively low voltage pacing pulses which are delivered to the patient's heart through low voltage, bipolar pacing leads, generally across spaced apart ring and tip electrodes thereof which are of opposite polarity. These pacing pulses assist the natural pacing function of the heart in order to prevent bradycardia. ICDs are sophisticated medical devices which are surgically implanted (e.g. abdominally or pectorally) in a patient to monitor the cardiac activity of the patient's heart, and to deliver electrical stimulation as required to correct cardiac arrhythmias which occur due to disturbances in the normal pattern of electrical conduction within the heart muscle. There is a need for improved cardiac stimulation devices which allow simplified implantation, as well as improved safety and effectiveness.

### SUMMARY

According to an aspect of the present inventive concepts, a system for stimulating cardiac tissue of a patient, the system comprises: a pulse generator comprising: a first transmission element for delivering wireless power; a stimulation assembly comprising: a flexible substrate; a second transmission element for receiving the wireless power from the first transmission element of the pulse generator; one or more electrodes attached to the substrate and configured to deliver electrical energy to cardiac tissue; and one or more microcircuits attached to the substrate and configured to deliver electrical energy to the one or more electrodes; and an algorithm comprising a fibrillation detection algorithm that is configured to determine when the one or more electrodes deliver the energy to the cardiac tissue.

In some embodiments, at least one of the one or more electrodes is configured to be implanted in a cardiac vein. The at least one electrode can be configured to be implanted in the vein of Marshall and/or the coronary sinus. The at least one electrode can comprise a first electrode configured to be implanted in the vein of Marshall, and a second electrode configured to be implanted in the coronary sinus.

In some embodiments, at least one of the one or more electrodes is configured to be implanted on an epicardial surface of the heart.

In some embodiments, the stimulation assembly comprises a first discrete portion including at least a first electrode and configured to be implanted at a first discrete location proximate the heart, and a second discrete portion comprising at least a second electrode and configured to be implanted at a second discrete location proximate the heart.

In some embodiments, the algorithm further comprises a pacing algorithm.

In some embodiments, the algorithm further comprises a cardioversion monitoring algorithm.

In some embodiments, the algorithm further comprises a post-cardioversion monitoring algorithm.

In some embodiments, the fibrillation detection algorithm comprises a bias toward false positive detection of fibrillation.

In some embodiments, the first and second transmission elements each comprise at least one antenna.

In some embodiments, the first and second transmission elements each comprise at least one coil.

In some embodiments, one of the one or more microcircuits comprises a first major axis, and the second transmission element comprises a second major axis, and the second major axis is longer than the first major axis.

In some embodiments, the first transmission element comprises a first major axis, and the second transmission element comprises a second major axis, and the first major axis is longer than the second major axis.

In some embodiments, the system further comprises a delivery device, and the delivery device comprises one or more devices constructed and arranged to implant at least the stimulation assembly in the patient.

In some embodiments, the system further comprises one or more sensors, and the one or more sensors are configured to record a physiologic parameter of the patient. The physiologic parameter can comprise one, two, or more parameters selected from the group consisting of: heart rate; blood pressure; respiration rate; blood glucose; blood gas level; pH; temperature; and combinations thereof. A first sensor of the one or more sensors can comprise an electrode of the one or more electrodes.

In some embodiments, the system further comprises a communication device configured to: transmit data to the stimulation assembly and/or the pulse generator; and/or receive data from the stimulation assembly and/or the pulse generator.

In some embodiments, the stimulation assembly further comprises at least one anchor. The at least one anchor can comprise an electrode of the one or more electrodes.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 is a schematic view of a system for delivering energy to electrically stimulate a patient's heart, consistent with the present inventive concepts.

FIG. 2 is an isometric cut-away view of a cardiac blood vessel with a stimulation assembly implanted along the vein of marshal and coronary sinus, consistent with the present inventive concepts.

FIGS. 3A-B are sectional anatomical views of an implanted stimulation assembly, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the terms “about” or “approximately” shall refer to ±20% of a stated value.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

The present inventive concepts generally relate to cardiac therapy and associated systems, and, more particularly, cardiac therapies and systems involving controlled delivery of electrical stimulations to heart tissue for treatment of atrial arrhythmias, other cardiac arrhythmias, and/or other undesired heart conditions.

A remedy for people with slowed or disrupted natural heart beating can be to implant a stimulation assembly of a system of the present inventive concepts. The stimulation assembly can comprise a cardiac pacing device that stimulates the heart to beat at regular rates.

The stimulation assembly can adapt its pulse rate to adjust the resultant heartbeats to the patient's level of activity, thereby mimicking the heart's natural beating. The system can modify that rate by tracking the activity at the sinus node of the heart and/or by responding to recordings from other sensors, such as sensors that monitor body motion and/or respiration rate.

Different pacing needs can be met by adjusting the programming of the system and/or by the selection of the implantation location of the electrodes that deliver the stimulation energy to the heart. The electrodes can be implanted within blood vessels of the heart, so that the electrodes can deliver energy to the portions of the muscle of the heart chamber requiring stimulation (e.g. requiring pacing).

In some patients, patch electrodes are placed on the exterior heart surface. With either type of electrode placement, it is important that the electrodes be attached to proper positions on the heart, such as to stimulate the appropriate muscles and produce desired contractions. Thus, it is desirable to properly locate the electrodes for maximum heart stimulation (e.g. optimized pacing effect) while causing minimal adverse impact to other physiological functions, such as blood circulation.

Some patients have hearts that occasionally go into fibrillation, where the heart has very rapid shallow contractions and, in the case of atrial fibrillation (AF), may not pump a sufficient amount of blood to support normal life function. Administration of a controlled electrical shock to the left atrium is often required to restore a normal rhythm, referred to as cardioversion. In some cases, an ablation procedure can be performed to restore a normal rhythm. Similar to a pacing device, the stimulation assembly of the present inventive concepts can be configured as an implanted defibrillator, where one or more sensors of the system senses a rapid heart rate (e.g. during fibrillation) and the stimulation assembly applies a relatively high energy electrical pulse to heart tissue (e.g. via one or more electrodes after and/or during the receipt of wireless power from a separate component of the system). When in a defibrillation mode, the stimulation assembly generates a much more intense electrical pulse than is used when in a pacing mode (e.g. a mode in which the energy delivered is configured to merely stimulate contractions of the heart).

In the treatment of a chronic cardiac condition, such as atrial arrhythmias, a challenge posed is that the patient typically is conscious and can potentially perceive any programmed electrical stimulation treatment being performed on their heart. Namely, one known method of electrical shock therapy for treating atrial (or ventricular) arrhythmia is to deliver a single burst of a relatively large amount of electrical current through the fibrillating heart of a patient. For a given atrial fibrillation episode, the minimum amount of energy required to defibrillate a patient's atrium is known as the atrial defibrillation threshold (ADFT). Generally speaking, the degree of pain, discomfort, and trauma caused to the conscious patient receiving electrical stimulation as the mode of therapy for a cardiac fibrillation generally will be a direct function of the amount of electrical energy delivered to the patient's heart to terminate a given fibrillation episode.

Therefore, it is desirable that the energy levels of electrical stimulating shocks delivered by the stimulation assembly of the present inventive concepts be reduced as much as practical, and ideally to below any significant pain threshold of the patient.

The systems of the present invention concepts can be configured to provide a method for terminating atrial fibrillation or at least to improve efficacy of stimulation energy delivery by bringing large regions of atrial tissue into phase-lock via a regimen of pacing level pulses alone. For example, a stimulation assembly of the present inventive concepts can deliver stimulation energy at a sufficient level to cause the tissue receiving the energy to phase-lock with the stimulation energy (e.g. the delivered stimulation energy overdrives the tissue and becomes the dominant driver of chamber contraction). The above and other objects, benefits and advantages can be achieved by the present inventive concepts as described herein.

A wireless, battery-less, sensing, and multi-site cardiac pacing device which includes a control circuit that has a fibrillation detection algorithm, which determines when a medical patient requires therapy, is disclosed herein. The device includes a wireless power transmitting component that, in response to the fibrillation algorithm determining that pacing therapy is indicated, causes a stimulation assembly to deliver stimulation to one, two or more cardiac sites. The stimulation assembly can include a distal anchor (e.g. a stent, such as a stent comprising an electrode for delivering stimulation energy and/or recording electrical activity) and a proximal anchor (e.g. a stent, such as a stent comprising an electrode for delivering stimulation energy and/or recording electrical activity). The stimulation assembly can include a substrate between the two anchors. The stimulation assembly can include multiple portions for implantation into blood vessels, such as for implantation at different locations in the patient. Operably mounted (e.g. electrically connected) to the substrate can be one or more microcircuits, and/or one or more electrodes, such as one or more electrodes that are pointing towards the left-atrium when the stimulation assembly is implanted in the patient. For example, the proximal stent anchor and the distal stent anchor can be implanted at different ends of the vein of Marshall and/or the coronary sinus. The microcircuits and energy-delivery electrodes can be placed in between. The microcircuits can comprise various electronic circuits and electronic componentry. The stimulation assembly, via one or more coils or antennas, can receive wireless power from a separate component of the system, such as a pulse generator as described herein. Upon receipt of the wireless power, the microcircuits can be selectively activated, and/or the electrodes can selectively deliver stimulation energy, such as in an order as defined by a pacing algorithm. The stimulation assembly can comprise a wireless power detector that is tuned to identify (and/or receive) the wireless power transfer. The microcircuits of the stimulation assembly can include a charging circuit that employs energy from wireless power received to charge a circuit which is configured as an electrical storage and/or dissipation device. A discharge circuit can respond to a control signal (e.g. issued by an algorithm) by applying the stored and/or harvested energy from the circuit to the electrodes, such as to produce a burst of multi-site pacing across the patient's left-atrial chamber to restore normal sinus rhythm.

The present inventive concepts relate to treatment modalities for atrial arrhythmias in which pacing is used to advantageously effect atrial fibrillation. ADFT energy requirements can be dramatically reduced or even eliminated while successfully defibrillating an arrhythmia (e.g. AF).

The present inventive concepts can include systems that deliver stimulation energy to one or more sites of the atrium, so as to maximize the extent of phase-locked area of atrial tissue. The instantaneous atrial pacing rate delivered at the pacing sites can be based on current AFCL data sensed in real time. The various pacing regimens disclosed herein can terminate atrial fibrillation (e.g. and maintain sinus rhythm), or, at the minimum, serve to significantly lower the energy requirements needed in an additional atrial defibrillation (ADF) shock-delivery tier of therapy. For example, after pacing has been conducted for a short period of time, ADF shocks can be delivered, if still needed, to terminate the atrial fibrillation episode.

In another embodiment, a method for terminating atrial fibrillation includes a multi-site pacing regimen in which venously-delivered multi-site burst pacing (i.e. multi-site burst pacing delivered by electrodes positioned in a vein of the heart) is conducted in an asynchronous manner, whereby the pacing is delivered at each of the multiple pacing sites as an interval train of pulses delivered at a predetermined coupling interval set proportional to a common AFCL value. This multi-site pacing regimen brings larger regions of fibrillating atrial tissue into phase-lock via delivery of energy pulses at multiple sites at a burst pacing level. Once phase-lock is obtained via such asynchronous venously-delivered multi-site pacing, the venously-delivered multi-site burst pacing continues until the atrial chambers are reset to a normal rhythm (e.g. the atrial chamber cells are reset to a normal rhythm). The selection of the common AFCL used for setting the pacing rates of the multiple pacing sites preferably is set to be equal to the minimum (i.e. shortest in a temporal sense) local AFCL value determined among the sensed local atrial sites. The local AFCL values can be determined by counting the number of depolarization wavefronts to enter the given atrial site over a selected period of time and then calculating the median or mean AFCL value from that information. This approach is especially useful where different locally sensed AFCL values vary from one another.

In another embodiment, a method for terminating atrial fibrillation including a multi-site burst pacing regimen in which venously-delivered multi-site pacing is conducted in an asynchronous manner, whereby the pacing is delivered concurrently to different micro pacing sites of the atrium, the vein of Marshall, and/or coronary sinus, such as in an independently controlled manner to procure an effect of overriding the refractory period of the action potential. The burst pacing is delivered at the multiple cardiac sites as an equal or unequal-interval train of pulses delivered at a predetermined coupling interval that is set proportional to the locally determined AFCL value (e.g. a value determined in real time). This multi-site burst pacing regimen also brings large regions of fibrillating atrial tissue into phase-lock via delivery of pacing level pulses alone. Once local phase-lock is obtained via such asynchronous venously-delivered multi-site pacing, if still necessary to terminate atrial fibrillation, the above process can be repeated.

In another embodiment, multi-site burst pacing of the atrium is conducted from an area outside the heart chamber that is adjacent to the atrial chamber, with a goal being to eventually entrain all atrial tissue by pacing alone. In this embodiment, the multi-site pacing devices can be implanted in the vein of Marshall and/or the coronary sinus (e.g. implanted to stimulate tissue in either or both of those locations). Alternatively or additionally, the multi-site pacing device can be implanted directly on the epicardial atrial surface, such as to deliver stimulate energy to one or more locations on that surface.

For purposes of this application, the following terms have the indicated meanings:

Capture: means pacing of the atria from one or more sites where each pacing stimulus results in a repeatable activation pattern of the entire atrium. The wavefronts originate at the pacing electrodes and the phase relationship between the pacing stimulus and the activation of each section of the atrial tissue remains constant throughout the pacing event.

Entrainment: means the same as capture.

Regional capture: pacing of the atrium from one or more sites where the stimulus results in wavefronts which depolarize only a portion of the myocardium surrounding the electrode or electrodes. The spatial extent of the depolarization caused by the pacing stimulus changes from beat to beat and occasionally may result in almost no propagated response. The wavefronts activating the captured region originate at, or near, the pacing electrode. The phase relationship remains constant between the pacing stimulus and activation of each section of myocardium within the region that is captured.

Phase-locking: pacing of the atrium from one or more sites which results in wavefronts that appear to be constant in phase with the pacing stimulus but where there does not appear to be a cause and effect relationship. The wavefronts do not appear to originate at the pacing sites and small changes in phase between the pacing stimulus and the activation of each section of a region occur over time. As a qualification, where EGM data on the atrium is limited, it is often difficult to differentiate between phase-locking and capture, as defined herein, and, for those cases, phase-locking terminology is used herein to refer to both capture and phase-locking.

Atrial Defibrillation Threshold or ADFT: The minimum amount of electrical energy required to defibrillate a fibrillating atrium of a patient.

Atrial Fibrillation Cycle Length or AFCL: the timing required between two consecutive depolarization wavefronts to traverse the same location.

Pacing Rate: also referred to herein as the S1-S1 interval, meaning the time intervals between delivery of successive pacing pulses.

Coupling Interval for Pacing Initiation or CIPI: means the time delay between the last local activation sensed, as the trigger, and the start thereafter of the first pulse of the pacing train.

Coupling Interval for Defibrillation Shock or CIDS: also referred to herein as the S1-S2 interval, meaning the time interval between the last pulse of a pulse train and the specific time thereafter when an ADF shock (i.e. the defibrillation trigger) is delivered.

Low potential gradient region of atrial tissue: the region in the atrium where the electric field lines generated by the current flowing between a pair of defibrillation electrodes positioned in the atrium are the least densely spaced. The location of this region can vary to the extent that the potential gradients generated by a defibrillation shock depend upon the particular lead configuration of the defibrillation electrodes in the atrium, the tissue conductivities, and torso geometry. The low potential gradient region can be located by measurement or intuitively.

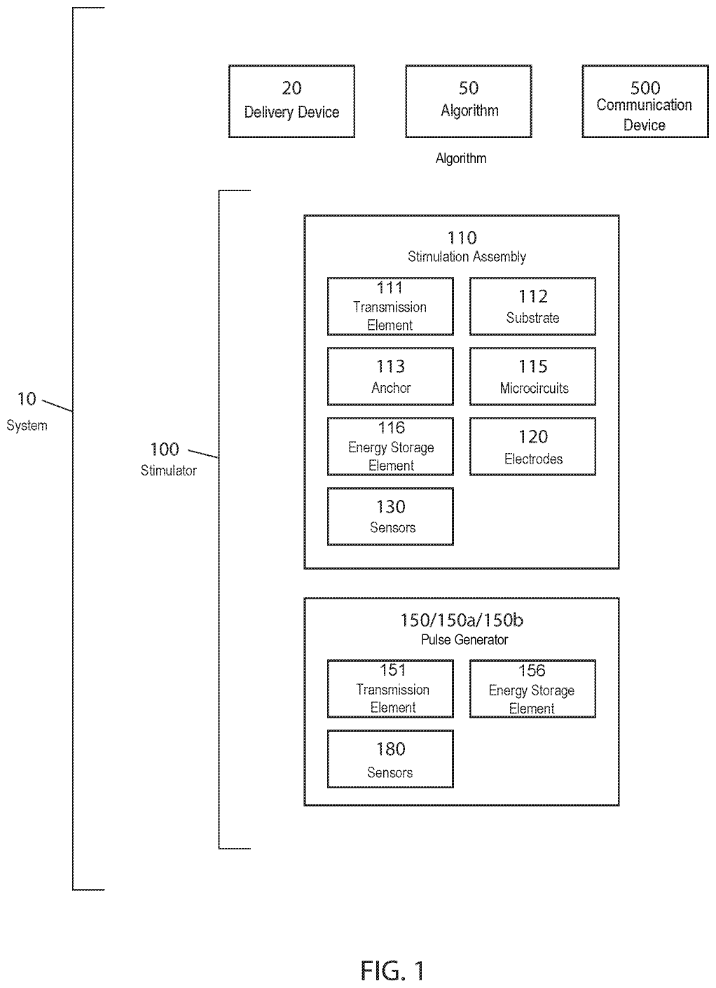

Referring now to FIG. 1, a schematic view of a system for delivering energy to electrically stimulate (e.g. pace and/or defibrillate) a patient's heart is illustrated, consistent with the present inventive concepts. System 10 comprises a tissue stimulating device, stimulator 100, which comprises stimulation assembly 110 for delivering electrical stimulation energy to heart tissue, and pulse generator 150 for supplying electrical energy to stimulation assembly 110. Pulse generator 150 can be configured to supply energy to stimulation assembly 110 via a wireless power transmission, as described herein. Stimulation assembly 110 is configured to be implanted within the patient's body (e.g. at a location within, and/or otherwise proximate a heart of a patient). Pulse generator 150 can be configured for positioning outside the patient's body, but relatively proximate stimulation assembly 110 (e.g. on the skin or otherwise within 1 meter of stimulation assembly 110 such as to accommodate power transfer to assembly 110). In some embodiments, pulse generator 150 is configured for implantation within the patient's body, such as at a location under the skin in the chest area of the patient. In some embodiments, pulse generator 150 comprises a first portion 150a for placement external to the patient, and a second portion 150b for implantation within the patient. System 10 can further comprise a data device, communication device 500 shown, such as for wirelessly communicating with stimulator 100.

Stimulator 100 can be configured to pace, defibrillate, and/or otherwise stimulate the heart by delivering electrical energy to multiple sites of the heart (e.g. multiple site stimulation provided simultaneously or sequentially). Stimulation assembly 110 can comprise an electronic assembly, microcircuits 115, which can include one or more integrated circuits, semiconductors, resistors, traces, and/or other electronic components. Microcircuits 115, and other components of assembly 110, can comprise flexible components, such as to ease placement within the vasculature of the heart and/or to accommodate motion of the heart after implantation in the heart. In some embodiments, stimulation assembly 110 does not include a battery or other element configured to store energy over an extended time period (e.g. at least one hour).

Stimulation assembly 110 can further include one or more electrode assemblies, electrodes 120, for contacting tissue of the heart and delivering electrical energy to the heart. Electrodes 120 can comprise stent-like scaffolds for engaging the wall of one or more blood vessels of the heart (e.g. one or more arteries and/or veins of the heart). In some embodiments, at least one electrode 120 is configured to be implanted within the vein of Marshall. In some embodiments, electrodes 120 comprise one or more electrodes for positioning on an endocardial or epicardial surface of the heart. In some embodiments, a set of multiple electrodes 120 are positioned to effectively stimulate a majority (e.g. the entire) of the posterior wall of the left atrium. Electrodes 120 can comprise electrodes with a ring-like geometry (e.g. full circumferential), or partial circumferential geometry (e.g. to approximate a partial circumferential portion of a blood vessel wall, such as to improve apposition with the blood vessel wall and/or to avoid imparting traumatic forces to the blood vessel wall).

Stimulation assembly 110 can comprise one or more antennas or other wireless transmission elements, transmission element 111, which can be configured to receive electromagnetic energy (i.e. power) from pulse generator 150 (e.g. from one or more antennas of pulse generator 150). Microcircuits 115 can comprise one or more energy capture and/or storage elements, ESE 116 shown. Each ESE 116 can be configured to capture, convert, and/or store the energy received by transmission element 111 (e.g. an element comprising one or more capacitors and/or batteries). In some embodiments, transmission element 111 comprises a component (e.g. one or more inductors) configured to inductively receive energy from pulse generator 150. In some embodiments, transmission element 111 comprises one or more antennas configured to transmit and/or receive data from pulse generator 150 and/or communication device 500. In some embodiments, transmission element 111 comprises a major axis that is greater than a major axis of microcircuits 115.

Pulse generator 150 can be configured to emit radiofrequency (RF) signals and/or other electromagnetic signals via one or more antennas, transmission element 151 shown. Transmission element 151 can be positioned relatively proximate transmission element 111 of stimulation assembly 110 such that power and/or data can be transmitted wirelessly between the two devices. Alternatively or additionally, transmission element 151 can comprise one or more inductors configured to inductively transmit energy to transmission element 111 of stimulation assembly 110. In some embodiments, transmission element 151 comprises one or more antennas configured to transmit and/or receive data from stimulation assembly 110 and/or communication device 500. In some embodiments, transmission element 151 comprises a major axis that is greater than a major axis of transmission element 111.

Pulse generator 150 can comprise one or more batteries, capacitors, and/or other energy storage elements, ESE 156 shown. In some embodiments, ESE 156 comprises a rechargeable element, such as when pulse generator 150 is configured to receive wireless transmissions of energy used to charge ESE 156. ESE 156 provides energy, via transmission elements 151 and 111, to microcircuits 115 of stimulation assembly 110, such as to supply power to the circuitry of microcircuits 115, and/or to deliver electrical energy to tissue, via electrodes 120, to stimulate the patient's heart.

As described herein, stimulation assembly 110 can be configured to deliver electrical energy to stimulate (e.g. pace) multiple cardiac sites. For example, stimulation assembly 110 can include a set of multiple electrodes 120, each configured to independently and controllably deliver electrical energy to a set of associated multiple cardiac sites.

System 10 can include one or more devices for implanting (e.g. percutaneously and/or surgically implanting) stimulation assembly 110, such as delivery device 20 shown. Delivery device 20 can comprise one or more catheters for percutaneously accessing the patient's vascular system, such as to implant stimulation assembly 110 in a blood vessel of the patient's heart. In some embodiments, delivery device 20 comprises one, two, or more devices that are collectively configured to implant two or more discrete portions of stimulation assembly 110 in two or more discrete locations of the patient's heart. In some embodiments, delivery device 20 comprises one or more devices for implanting all or a portion of pulse generator 150. In some embodiments, delivery device 20 comprises a laparoscopic implantation tool.

Stimulation assembly 110 can comprise one or more substrates, substrate 112 shown, such as to operably connect (e.g. electrically, mechanically, and/or fluidically connect) various components of stimulation assembly 110. Substrate 112 can comprise one or more flexible circuit boards, and it can include one or more semiconductors, sensors, and/or other passive and/or active components.

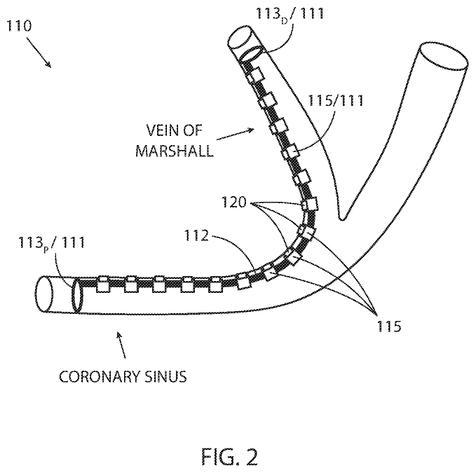

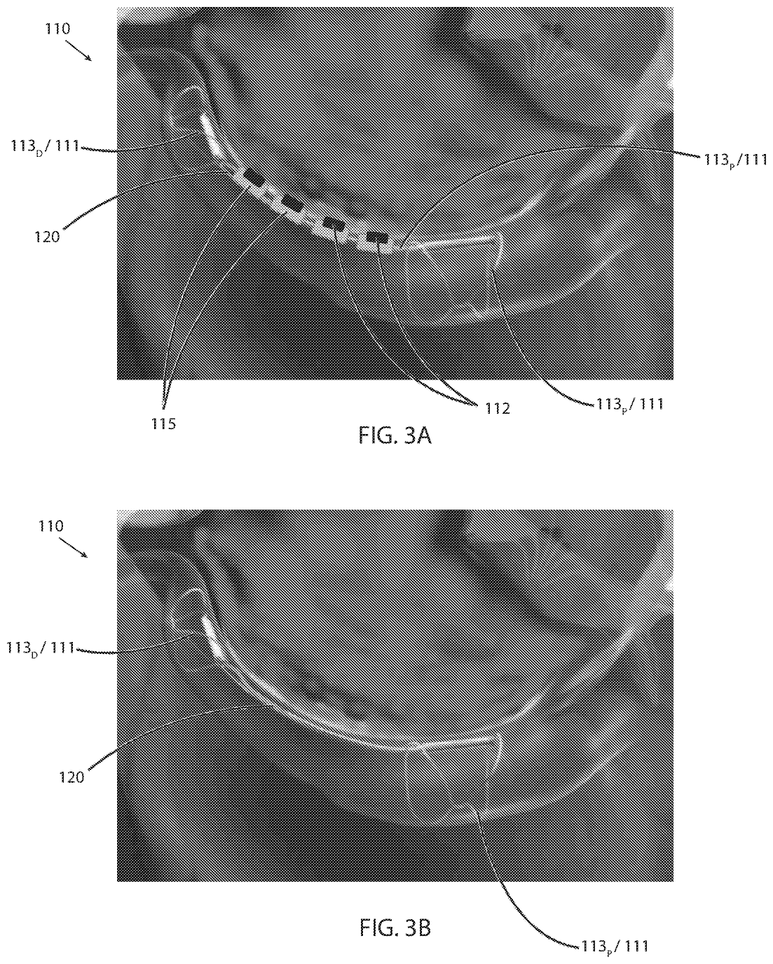

Stimulation assembly 110 can comprise one or more anchoring elements, anchor 113 shown. In some embodiments, stimulation assembly 110 comprises at least two anchors 113, such as an anchor positioned on both a proximal portion and distal portion of assembly 110, such as is described herebelow in reference to FIGS. 2 and 3A-B. Anchor 113 can comprise a ring-like structure when deployed. Anchor 113 can comprise a self-expanding structure, and/or a plastically deformable structure, such as to support an expansion to engage blood vessel walls. Alternatively or additionally, anchor 113 can comprise a barb, hook, or other tissue-engaging element, such as to secure stimulation assembly 110 to an endocardial and/or epicardial surface of the patient's tissue. In some embodiments, anchor 113 comprises a stent-like structure, extending along at least a portion of substrate 112. For example, anchor 113 can comprise a construction similar to a stent, and substrate 112 can be fixedly attached along the length of anchor 113. In some embodiments, one or more anchors 113 comprise a transmission element 111 (e.g. an anchor 113 and transmission element 111 are the same component or otherwise integrated together, such as when transmission element 111 comprises a ring-like structure configured both as an antenna and as a blood vessel anchor).

System 10 can include various sensors. For example, stimulation assembly 110 can comprise one or more sensors, sensor 130 shown. In some embodiments, sensor 130 comprises one or more electrodes 120 (e.g. an electrode configured to both deliver electrical energy and record electrical signals of the heart). In some embodiments, one or more microcircuits 115 comprise a sensor 130. Pulse generator 150 can comprise one or more sensors, sensor 180 shown. Sensors 130 and/or 180 can each comprise one, two, ore more sensors that are configured to record an electrocardiogram (EKG) of the patient's heart activity. Sensors 130 and/or 180 can each comprise one, two or more sensors configured to record a physiologic parameter of the patient, such as a parameter selected from the group consisting of: heart rate; blood pressure; respiration rate; blood glucose; blood gas level; pH; temperature; and combinations of these. In some embodiments, sensors 130 and/or 180 can comprise two or more sensors configured to record two or more of these physiologic parameters.

As described hereabove, pulse generator 150 can comprise multiple discrete components, such as a first portion 150a that is positioned outside of the patient, and a second portion 150b that is implanted in the patient. Second portion 150b, via its transmission elements 151b, can wirelessly transfer power to stimulation assembly 110. In some embodiments, first portion 150a wirelessly transfers power to either or both of stimulation assembly 110 and second portion 150b.

Also as described hereabove, system 10 can include one or more devices for communicating with another component of system 10, such as communication device 500 shown. Communication device 500 can be configured to wirelessly communicate (e.g. via Bluetooth or other wireless communication arrangement) to deliver data to a system 10 component (e.g. deliver programming data and/or other information to stimulation assembly 110 and/or pulse generator 150), and/or to receive data from a system 10 component (e.g. receive patient physiologic information and/or system 10 use information from stimulation assembly 110 and/or pulse generator 150).

System 10 can comprise one or more algorithms, algorithm 50 shown. Algorithm 50 can comprise one or more algorithms that are integrated into one, two, or more of: pulse generator 150; stimulation assembly 110 (e.g. integrated into one or more microcircuits 115); communication device 500; and/or another component of system 10. Algorithm 50 can be configured to analyze patient information, such as at least patient EKG information, such as to determine when stimulation energy should be delivered by stimulation assembly 110 to the patient to pace and/or defibrillate the patient. In some embodiments, algorithm 50 is configured to determine parameters of the stimulation waveform to be delivered (e.g. to determine amplitude, frequency, pulse-width, and/or other stimulation waveform information). In some embodiments, algorithm 50 is configured to analyze data recorded by two sensors (two or more from a collection of sensors 130 and/or sensors 180). In some embodiments, algorithm 50 comprises a bias to perform a “safer” treatment when recordings from two or more sensors include conflicting data. For example, the bias can cause system 10 to tend to stimulate (e.g. a bias towards a false positive stimulate indication), such as when information from one sensor indicates stimulation is needed, and bias from another sensor indicates stimulation is not needed. In other embodiments, algorithm 50 comprises a bias towards false negatives (e.g. not stimulating unless both sensor signals are indicative of stimulation being required).

In some embodiments, algorithm 50 comprises a “fibrillation detection algorithm”, such as when system 10 is configured to deliver stimulation energy (e.g. pacing and/or defibrillation energy) when the fibrillation detection algorithm detects that the patient's heart is in fibrillation (e.g. atrial fibrillation). The fibrillation detection algorithm can include a bias, such as a bias toward false positives (a bias that tends to cause stimulation assembly 110 to deliver energy when fibrillation or other arrhythmia is not present, versus not delivering stimulation energy when fibrillation or other arrhythmia is present). In some embodiments, algorithm 50 comprises a “stimulation waveform creation algorithm”, also referred to herein as a “pacing algorithm”, which can be configured to determine one or more parameters of stimulation energy to be delivered (e.g. amplitude, frequency, pulse width, and the like), such as to determine an optimized and/or otherwise desired set of stimulation parameters to properly pace and/or defibrillate a patient's heart. In some embodiments, algorithm 50 comprises a first algorithm comprising a fibrillation detection algorithm, and a second algorithm comprising a pacing algorithm. In these embodiments, the fibrillation algorithm can identify when the patient is in fibrillation (e.g. AF), and then the pacing algorithm can be initiated to cause a desired stimulation pattern (e.g. one or more stimulation waveforms) to be delivered by stimulation assembly 110 to treat the fibrillation. In some embodiments, algorithm 50 comprises a “cardioversion monitoring algorithm” configured to determine when the application of stimulation energy by stimulation assembly 110 has caused the patient to revert to a normal rhythm (e.g. sinus rhythm). Once the normal rhythm is detected, delivery of stimulation energy can be stopped. In some embodiments, algorithm 50 comprise a “post-cardioversion monitoring algorithm” which is implemented after a recovery to a normal rhythm (e.g. sinus rhythm) has been achieved (sometime after an arrhythmia occurred and therapeutic stimulation energy has been delivered). This post-cardioversion monitoring algorithm can be configured to identify an adverse condition (e.g. an arrhythmia) that may result soon after recovery to a normal rhythm (e.g. the post-cardioversion algorithm can comprise a more sophisticated and/or otherwise modified version of a fibrillation detection algorithm).

In some embodiments, algorithm 50 comprises a pacing algorithm that determines a set of stimulation waveform delivery patterns that change over the course of stimulation. For example, during the initial stages of stimulation energy delivery, a stimulation waveform at a first frequency can be delivered, and over time, the frequency can be decreased, such as to drive contraction at a higher rate than normal during this initial stage, and slow the contraction to a normal rate (e.g. sinus rhythm) over time.

In some embodiments, algorithm 50 comprises an algorithm that is of similar construction and arrangement as that described in U.S. patent Ser. No. 13/738,626, titled “Atrial Fibrillation Classification Using Power Measurement”, filed Jan. 10, 2013.

In some embodiments, system 10 and/or one or more of its components are of similar construction and arrangement as the system and components described in International PCT Patent Application Serial Number PCT/US2019/062443, titled “Systems and Methods for Controlling Wirelessly Powered Leadless Pacemakers”, filed Nov. 20, 2019.

Referring to FIG. 2, an anatomical sectional view of a stimulation assembly implanted in a patient's heart is illustrated, consistent with the present inventive concepts. Stimulation assembly 110 of FIG. 2 is shown implanted in the patient's coronary sinus, extending into the vein of Marshall. In other embodiments, at least a portion of stimulation assembly 110 is positioned in different blood vessels (e.g. arteries and/or veins) of the patient's heart, and/or on an endocardial and/or epicardial surface of the patient's heart. Stimulation assembly 110 can be of similar construction and arrangement to stimulation assembly 110 described herein in reference to FIG. 1. Stimulation assembly 110 can be configured to wirelessly receive power from a separate component of system 10 (e.g. from pulse generator 150, not shown), and to use the received energy to stimulate heart tissue. Additionally or alternatively, stimulation assembly can be configured to use the received energy to power one or more components of stimulation assembly 110, for example to power a sensor 130 to record a physiologic parameter of the patient, and/or to power transmission element 111 to transmit the recorded data back to pulse generator 150 and/or communication device 500. In some embodiments, algorithm 50 (described herein) can determine a stimulation parameter based on the recorded information, and pulse generator 150 can transmit power and/or information to stimulation assembly 110 in an amount sufficient to provide the determined stimulation. Stimulation assembly 110 of FIG. 2 includes multiple microcircuits 115 (fifteen shown), each physically and electrically attached to a flexible printed circuit board, substrate 112. Stimulation assembly 110 further comprises multiple electrodes 120 (fifteen shown), each positioned up against the wall of the cardiac blood vessel such as to deliver the stimulating energy to heart tissue. Stimulation assembly 110 further comprises two anchor elements, proximal anchor 113P and distal anchor 113D shown. Anchors 113 can be configured to radially expand during deployment (e.g. via a resilient bias or plastic deformation), such as to frictionally engage the wall of the blood vessel into which anchors 113 are inserted, stabilizing stimulation assembly 110 in the patient's heart. Each anchor 113 can be of similar construction and arrangement to that of a vascular stent. Once expanded, each anchor 113 can comprise a relatively circular shape that engages the wall of the blood vessel without significantly obstructing the blood vessel (e.g. without impinging the flow of blood through the blood vessel). Stimulation assembly 110 further comprises one or more transmission elements 111, such as a transmission element 111 integrated into a microcircuit 115a, a transmission element 111 integrated into proximal anchor 113_{P}, and/or a transmission element 111 integrated into distal anchor 113_{D}, each as shown.

One or more microcircuits 115 can comprise a structure configured to be in (e.g. expand into) a relatively circular shape when implanted (e.g. when implanted in a vein, artery, or other blood vessel of the patient's heart). Once implanted and in the relatively circular shape, a microcircuit 115 can engage the wall of a blood vessel without significantly obstructing the blood vessel (e.g. without impinging the flow of blood through the blood vessel). Each microcircuit 115 can comprise a tissue-engaging portion (e.g. a vessel wall-engaging portion) that is of similar construction and arrangement to that of a vascular stent. Such vascular stents generally have a construction that exerts radial force and is initially collapsed to a relatively small diameter enabling the stent to pass freely through a blood vessel of a patient prior to implantation.

The procedure for implanting stimulation assembly 110 can be similar to that used for conventional vascular stents. For example, delivery device 20 (described in reference to FIG. 1 herein) can be of similar construction and arrangement as a standard stent delivery catheter used in stent implantation procedures. During the procedure in which stimulation assembly 110 is implanted, one or more portions of stimulation assembly (transmission elements 111, substrate 112, anchor 113, microcircuits 115, and/or electrodes 120) can be in a collapsed (e.g. radially collapsed) state. Delivery device 20, including stimulation assembly 110, is inserted through an incision in a vein or artery near the skin of a patient (e.g. through an introducer or other percutaneous access device) and advanced through the vascular system to the appropriate location within or otherwise proximate the heart. In some embodiments, at least electrodes 120 of stimulation assembly 110 are positioned in a cardiac blood vessel (e.g. a cardiac vein) that is adjacent to a tissue portion of the heart muscle where stimulation should be applied. Delivery device 20 can comprise a balloon for deploying (e.g. plastically deforming) at least a portion of stimulation assembly 110 (e.g. anchors 113), and/or it can include a retractable sheath for deploying (e.g. allowing self-expansion of) stimulation assembly 110 (e.g. when stimulation assembly 110 comprises one or more nickel-titanium alloy-based or other self-expanding components).

Deployment (e.g. expansion) of stimulation assembly 110 can be configured to cause a slight radial expansion of the walls of the associated blood vessel. This slight expansion of the blood vessel wall, and a low-profile design of each component of stimulation assembly 110 (e.g. low-profile design of microcircuits 115 and other components of stimulation assembly 110) allows unimpeded blood to flow through the segment of the blood vessel into which stimulation assembly 110 is implanted.

After stimulation assembly 110 is deployed, delivery device 20 and any other implantation devices are removed from the patient, and the incision is closed. Stimulation assembly 110 remains in the blood vessel, and it can be configured to receive power for stimulating the patient's heart via wireless power transmission. For example, stimulation assembly 110 can be void of a power supply sufficient to provide energy to stimulate the heart, and/or it can be void of a wired connection (e.g. avoiding wires that may be susceptible to breaking) to provide the energy.

As described herein, stimulation assembly 110 comprises various components configured to receive power and/or data such as to deliver stimulation energy to a patient's heart, such as when stimulation assembly 110 has been surgically and/or percutaneously delivered to a location within and/or proximate the patient's heart. Stimulation assembly 110 can be configured to execute commands (e.g. as received from pulse generator 150 and/or communication device 500, each not shown), such as commands that are executed via control circuitry of microcircuits 115. Stimulation assembly 110 can be configured to monitor one or more patient physiologic parameters (e.g. as measured by a sensor 130 recording EKG signals and/or other physiologic parameter of the patient as described herein), and to deliver stimulation (e.g. pacing and/or defibrillation) based on an analysis of the monitored parameters (e.g. an analysis performed by algorithm 50 as described herein). Stimulation assembly 110 can comprise one or more transmission elements 111 (e.g. antennas) for receiving power from a separate component of system 10, such as from transmission elements 151 (e.g. antennas) of pulse generator 150 (e.g. an implanted and/or externally positioned pulse generator 150). Stimulation assembly 110 uses the received energy to power microcircuits 115, power any sensors 130 requiring power, and/or to transmit energy to tissue (via electrodes 120), such as to pace or defibrillate the heart. Transmission element 151 (and/or microcircuits 115) can comprise circuitry configured to tune an antenna of transmission element 151 to efficiently receive power transmissions provided to stimulation assembly 110. Upon detecting the transmitted power (e.g. via a signal detector of microcircuits 115), stimulation assembly 110 converts the energy of that transmission into an electric current that can be delivered by one, two, or more electrodes 120 to tissue. Those electrodes 120 form an electric circuit path with the patient's heart tissue that effectively stimulates (e.g. paces and/or defibrillates) the patient's heart.

Stimulation assembly 110 can comprise one, two or more discrete portions, each portion comprising one, two, or more electrodes 120. Each portion of stimulation assembly 110 can receive power in either an asynchronous and/or synchronous communication arrangement. Each portion can deliver stimulation energy to tissue (e.g. via the electrodes 120 of that portion), in a synchronous and/or asynchronous arrangement with one or more other portions of stimulation assembly 110.

Electrodes 120 can be positioned in one or more cardiac blood vessels at locations that are directly associated with the specific muscles requiring therapy (e.g. requiring pacing, defibrillating, and/or other stimulation).

In some embodiments, stimulation assembly 110 comprises multiple microcircuits 115, and each of these microcircuits 115 comprises one, two, or more electrodes 120. In these embodiments, each microcircuit 115 and its associated electrodes can be configured to independently deliver stimulation energy to the tissue proximate (e.g. adjacent) the electrodes 120, such as in a simultaneous or sequential delivery scheme with other microcircuit 115 and associated electrode 120 combinations. Each microcircuit 115 can include a unique identifier (ID), such as to receive specific communications from pulse generator 150 and/or communication device 500. Each set of electrodes 120 can be positioned at a different cardiac location, such as one or more locations selected from the group consisting of: cardiac vein; cardiac artery; endocardial surface of a cardiac chamber; epicardial surface of the heart; and combinations of one or more of these.

In some embodiments, a first set of one or more microcircuits 115 receive power from a first set of one or more transmission elements 111 (e.g. antennas), and a second set of one or more microcircuits 115 receive power from a second set of one or more transmission elements 111. In these embodiments, the first and second sets of transmission elements 111 can receive power from the different power transmissions (e.g. independent power transmissions from pulse generator 150, such as two or more power transmissions comprising different frequencies or other unique features), and/or from the same power transmission (e.g. a single power transmission from pulse generator 150). Pulse generator 150 and its transmission elements 151 (e.g. antennas) can be configured to provide independent power transmission signals, and/or power transmission signals that are efficiently received by one or more sets of transmissions elements 111 (e.g. and provide energy to one or more associated microcircuits 115 and/or electrodes 120). Pulse generator 150 can be configured to specify (e.g. determine) the duration and frequency of the power transmission signal, such as to correspondingly control the stimulation energy provided by stimulation assembly 110 to tissue (e.g. control the duration and/or frequency of stimulation energy delivery at one or more tissue locations). For example, in this configuration different portions of the heart muscle can be stimulated independently (e.g. in a simultaneous and/or sequential arrangement) by varying the wireless power transmission emitted from pulse generator 150 to correspond to the frequency to which the portion of stimulation assembly 110 in a given location is tuned or timed (e.g. to correlate to the tuning of transmission element 111 and microcircuits 115, and/or to correlate to the timing of stimulation energy to be delivered by electrodes 120 to tissue). Multiple stimulating portions (e.g. microcircuits 115 and associated electrodes 120) of stimulation assembly 110 can be activated in a given sequence by producing a series of power transmissions at different frequencies and/or different timing schemes. This arrangement enables stimulation assembly 110 to deliver stimulation energy (e.g. pacing energy) to produce a normal contraction of the heart chambers, such as to increase cardiac efficiency. In some embodiments, stimulation assembly 110 is configured to initially pace the heart at a higher than normal rate, and to decrease to a normal rate over time.

The stimulation energy delivered by the electrodes 120 of stimulation assembly 110 travels along the proper tissue pathways by means of a complex cell reaction that allows each cell to activate the cell next to it, stimulating it, such as to “pass along” the electrical signal in an orderly manner. As cell after cell rapidly transmits the electrical charge, the entire heart chamber contracts in one coordinated motion.

As described herein, system 10 can be configured to defibrillate the patient's heart, such as when stimulation assembly 110 is configured to function as an internal cardiac defibrillator (ICD). In some embodiments, pulse generator 150 comprises at least a portion (e.g. portion 150a described herein) which is implanted in the patient's body, and is configured as at least a portion of an ICD, such as when pulse generator 150 includes a battery (e.g. ESE 156) and control circuitry, and delivers power (e.g. wirelessly) to stimulation assembly 110 to defibrillate the patient's heart. Stimulation assembly 110 can include circuitry (e.g. microcircuits 115 and/or transmission elements 111) which communicate with the implanted, ICD-based, pulse generator 150.

Similar to the pacing arrangement described herein, when configured to defibrillate the heart, pulse generator 150 can comprise an implanted device (e.g. implanted under the skin, proximate the heart of the patient), an external device, or both, that wirelessly transmits power to stimulation assembly 110. System 10 (e.g. pulse generator 150 and/or stimulation assembly 110) can include algorithm 50, which can be configured to detect an irregular heart rate (e.g. as determined by one or more electrodes 120, sensors 130, sensors 180, and/or other sensors of system 10), and deliver stimulation energy to pace, defibrillate, and/or otherwise therapeutically stimulate the patient's heart. For example, once the irregular heartbeat is detected by algorithm 50, pulse generator 150 can begin wireless power transmissions to stimulation assembly 110, and stimulation assembly 110 can begin delivering the stimulation energy to the patient's heart.

As described herein, stimulation assembly 110 comprises one or more coils or antennas, transmission element 111, that receives power transmissions from pulse generator 150 (e.g. power transmissions from transmission element 151 of pulse generator 150). Transmission elements 111, microcircuits 115, and/or other components of stimulation assembly 110 can be tuned to one or more frequencies of the power transmissions and can deliver the received energy to microcircuits 115 (e.g. including one or more sensors 130) and/or electrodes 120. The charging circuit uses the signal energy to charge capacitor(s). In some embodiments, microcircuits 115 include one or more capacitors that store energy received via transmission elements 111. When the charge on the one or more capacitors is sufficient to produce a pacing, defibrillating, and/or other desired stimulation pulse, a discharge circuit of microcircuit 115 can “dump” the charge to the associated electrodes 120. One or more electrodes 120 configured to deliver electrical current can be connected by wires and/or conductive traces of substrate 112 to an electrode 120 configured as a grounding electrode, thereby providing a return pole to complete an electrical circuit (through tissue) for the stimulation pulse.

This configuration results in the application of an electrical pulse across a first electrode 120, as well as a second, third and/or fourth electrode 120, which “shocks” the patient's heart muscle in order to restore (e.g. eventually restore) a normal cardiac rhythm. Delivery of energy from multiple electrodes 120 at multiple cardiac locations (e.g. multiple electrodes 120 at one, two or more discrete portions of stimulation assembly 110) provides a greater dispersion of the stimulation energy and avoids a local discharge.

In some embodiments, the power transmission sent to stimulation assembly 110 by pulse generator 150 can comprise a duration that is sufficient to charge one or more energy storage capacitors of stimulation assembly 110 to a level necessary to deliver a desired pulse of stimulation energy. In some embodiments, pulse generator 150 periodically sends a power transmission to stimulation assembly 110. This power transmission does not necessarily cause stimulation assembly 110 to deliver stimulation energy to the patient's heart as it can be used merely to maintain a requisite charge on an energy storage capacitor of a microcircuit 115. This minimum energy storage arrangement ensures that the capacitor will be nearly fully charged and/or properly cycled when stimulation energy should be delivered and shortens the time between receipt of an energy delivery signal and the actual delivery of the stimulation energy to the heart muscle. In these embodiments, pulse generator 150 can transmit an encoded control power transmission signal when the patient requires pacing and/or defibrillation (i.e. when pacing and/or defibrillation energy should be delivered). Stimulation assembly 110 responds to that encoded control signal by triggering one or more microcircuits 115 to deliver the stimulation energy via the one or more associated electrodes 120 to the patient's heart.

Referring now to FIGS. 3A and 3B, sectional anatomical views of stimulation assemblies comprising proximal and distal anchors and implanted in a blood vessel are illustrated, consistent with the present inventive concepts. In FIG. 3A, stimulation assembly 110 comprises proximal and distal anchors 113_{P} and 113o respectively, which are connected to each other via substrate 112 (e.g. a flexible printed circuit board). Operably connected to substrate 112 are microcircuits 115 (four shown), as well as electrodes 120 (four shown). Stimulation assembly 110 of FIG. 3B has microcircuits 115 and electrodes 120 removed for illustrative clarity. Stimulation assembly 110 of FIGS. 3A-B can be of similar construction and arrangement to stimulation assemblies 110 of FIGS. 1 and/or 2. Stimulation assembly 110 can be implanted in a vein or artery of the heart, such as the vein of Marshall.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which is defined in the accompanying claims.

1. A system for stimulating cardiac tissue of a patient, the system comprising:
a pulse generator comprising:
a first transmission element for delivering wireless power;
a stimulation assembly comprising:
a flexible substrate;
a second transmission element for receiving the wireless power from the first transmission element of the pulse generator;
one or more electrodes attached to the substrate and configured to deliver electrical energy to cardiac tissue; and
one or more microcircuits attached to the substrate and configured to deliver electrical energy to the one or more electrodes;
and
an algorithm comprising a fibrillation detection algorithm that is configured to determine when the one or more electrodes deliver the energy to the cardiac tissue.

2-21. (canceled)
